(19) United States
(12) Patent Application Publication
(10) Pub. No.: US 2024/0139405 A1
(43) Pub. Date: May 2, 2024

(54) AN ACCESSORY FOR A MEDICAL DEVICE TO DISINFECT A SKIN SURFACE

(71) Applicant: Becton, Dickinson and Company, Franklin Lakes, NJ (US)

(72) Inventors: Jeremy GARTNER, Closter, NJ (US); Maryann FAM, Secaucus, NJ (US); Peter GIRGIS, East Brunswick, NJ (US)

(73) Assignee: Becton, Dickinson and Company, Franklin Lakes, NJ (US)

(21) Appl. No.: 18/270,183

(22) PCT Filed: Dec. 14, 2021
(86) PCT No.: PCT/US2021/063368

§ 371 Date: Jun. 28, 2023

Related U.S. Application Data
(60) Provisional application No. 63/132,675, filed on Dec. 31, 2020.

(51) Int. Cl. A61M 5/00; A61M 5/32

(52) U.S. Cl. CPC A61M 5/001; A61M 5/3202; A61M 5/3243

(57) ABSTRACT

An accessory (60) configured to be attached to a medical device (10) to disinfect a skin surface or a portable medical device surface (54). The accessory (60) comprising a plurality of disinfecting materials (68), and a mounting mechanism (62) that is configured to attach and detach the accessory (60) to the medical device (10).

### RELATED APPLICATIONS

This application is related to U.S. Nonprovisional application Ser. No. 16/988,576, filed Aug. 7, 2020 which is hereby incorporated by reference in its entirety. This application also claims the benefit under 35 U.S.C. § 119(e) of U.S. Provisional Application Ser. No. 63/132,675, filed Dec. 31, 2020, the entire content of which is hereby incorporated by reference.

### FIELD OF THE INVENTION

The present invention relates to an accessory detachably connected to a medical device for disinfecting a portion of the medical device (a portable medical device surface, for example) and/or a skin surface at an injection site prior to administering medicament.

### BACKGROUND OF THE INVENTION

Insulin and other injectable medications are commonly given with a medical device, such as a drug delivery device or a medication delivery pen, whereby a disposable pen needle is attached to facilitate drug container access and allow fluid egress from the container, through the needle and into the patient.

As technology and competition advance, driving the desire for shorter, thinner, less painful, and more efficacious injections, the design of the drug delivery devices such as a pen needle and parts thereof becomes more and more important. Designs need to proactively address ergonomically improving injection technique, injection depth control and accuracy, the ability to be safely used and transported for disposal, sterilization, disinfection, and protection against misuse while maintaining the ability to be economically manufactured on a mass production scale.

Drug delivery devices, such as the exemplary medication delivery pen 10 shown in FIGS. 1 and 2, can be designed for subcutaneous as well as intradermal injections, and typically comprise a dose knob/button 22, an outer sleeve or housing 11, and a cap 50. The dose knob/button 22 allows a clinician or patient to set the dosage of medication to be injected. The housing 11 is gripped by the user when injecting medication. The cap 50 can be used by the user to securely hold the medication delivery pen 10 in a shirt pocket, purse or other suitable location and provide cover/protection from accidental needle injury. The cap 50 is also used to cover a septum 18 of the medicament cartridge 16 in the medication delivery pen 10 before and after use. Otherwise, the septum 18 would be exposed.

FIG. 2 is an exploded view of the medication delivery pen 10 of FIG. 1. The dose knob/button 22 has a dual purpose and is used both to set the dosage of the medication to be injected and to inject the dosed medicament via the leadscrew 12 and plunger/stopper 14 through the medicament cartridge 16, which is attached to the medication delivery pen 10 through a body 20. In standard medication delivery pens, the dosing and delivery mechanisms are all found within the housing 11 and are not described in greater detail here as they are understood by those knowledgeable of the prior art.

For operation, the medication delivery pen 10 is attached to a pen needle comprising a needle/cannula 30, a septum penetrating cannula 32 and a hub 34. Specifically, a distal movement of the plunger or stopper 14 within the medicament cartridge 16 causes medication to be forced into the needle 30 of the hub 34. The medicament cartridge 16 is sealed by the septum 18, which is punctured by the septum penetrating needle cannula 32 located within the hub 34. The hub 34 is preferably screwed onto the body 20, although other attachment means can be used.

To protect a user from accidental needle sticks, or anyone who handles the pen needle, an outer cover 38, which attaches to the hub 34, covers the hub 34. An inner shield 36 covers the patient needle 30 within the outer cover 38. The inner shield 36 can be secured to the hub 34 to cover the patient needle 30 by any suitable means, such as an interference fit or a snap fit. The outer cover 38 and the inner shield 36 are removed prior to use.

The medicament cartridge 16 is typically a glass tube or vial sealed at one end with the septum 18 and sealed at the other end with the stopper 14. The septum 18 is pierceable by a septum penetrating cannula 32 in the hub 34, but does not move with respect to the medicament cartridge 16. The stopper 14 is axially displaceable within the medicament cartridge 16 while maintaining a fluid tight seal.

Existing medication delivery pens are disclosed in U.S. Patent Application Publication Nos. 2006/0229562 to Marsh et al., which was published on Oct. 12, 2006, and 2007/0149924 to R. Marsh, which was published on Jun. 28, 2007, the entire contents of both of which are hereby incorporated by reference for this purpose.

Medical devices such as medication delivery pens 10 are typically prepared for use by disinfecting the septum 18 with an alcohol swab prior to attaching the pen needle for medication delivery and disinfecting a skin surface at an injection site with an antiseptic wipe prior to administering medicament. However, challenges arise to consistently and accurately disinfect the medication delivery pens 10 and the skin surface for safe patient care. Carrying alcohol swabs and/or antiseptic wipes with the medication delivery pens 10 can be burdensome and inconvenient for a user. Further, the alcohol swabs and/or antiseptic wipes have shelf-life limitations and are typically for single-use only. In certain circumstances, the septum 18 may not be properly disinfected prior to use. Consistently following best disinfection practices is not always adhered to and it can be difficult to ensure for each and every disinfection. Thus, an improved disinfecting device and process for use with medical devices such as medication delivery pens 10 is desired.

### SUMMARY OF THE INVENTION

It is an aspect of the present invention to provide an accessory detachably connected to a medical device that is capable of disinfecting a skin surface at an injection site alone or in combination with disinfecting a medical device or a portion thereof, such as a septum surface. Such a configuration improves workflow and convenience of users using various medical devices such as medication delivery pens, syringes, patch pumps, safety pens, safety devices, and insulin vials. Poor injection practice is minimized since the user is no longer relied upon to disinfect the skin surface, septum or other exposed surface or portion of the medical device (a portable medical device surface) with an alcohol swab or antiseptic wipe.

In an aspect of the present invention, the medical device can be configured to be connected to an accessory and automatically disinfect the septum and/or other exposed surface or portion, thus saving time. Moreover, the skin surface is more conveniently disinfected either simultaneously or alternately with respect to disinfecting of the septum to improve workflow and optimize time. Disinfecting the medical device and the skin surface with the accessory is also more controlled or automated to satisfy high accuracy and performance requirements. The user no longer needs to carry alcohol swabs and/or antiseptic wipes for the medical device and/or the skin surface.

It is another aspect of the present invention to provide an accessory that is attachable to a device to disinfect the skin surface prior to injection. Such an accessory provides disinfecting material to disinfect a skin surface prior to needle injection of the device. The accessory is also capable of carrying a plurality of disinfecting materials and/or multiple disinfectant applications for convenient use. The plurality of disinfecting materials can be successively used either manually or automatically. Specifically, the accessory can automate discarding a used disinfecting material while automatically preparing an unused disinfecting material for disinfection. The accessory is also adaptable to various products, including medical devices, and is configured to attach and detach for versatile and convenient use.

The foregoing and/or other aspects of the present invention can be achieved by providing an accessory configured to be attached to a medical device to disinfect a skin surface, the accessory comprising a plurality of disinfecting materials, and a mounting mechanism that is configured to attach and detach the accessory to the medical device.

The foregoing and/or other aspects of the present invention can further be achieved by providing an accessory configured to be attached to a medical device to disinfect a skin surface, the accessory comprising an applicator providing a disinfecting material, and a mounting mechanism that is configured to attach and detach the applicator to the medical device.

The foregoing and/or other aspects of the present invention can also be achieved by providing an accessory configured to be attached to a medical device to disinfect a skin surface, the accessory comprising a cylinder wrapped by a strip of disinfecting materials, and a mounting mechanism that is configured to attach and detach the cylinder to the medical device, wherein the strip of disinfecting material is unwrapped to disinfect the skin surface.

The foregoing and/or other aspects of the present invention can additionally be achieved by providing a method for disinfecting a skin surface, the method comprising attaching an accessory to a medical device, activating the accessory to provide an unused disinfecting agent, disinfecting the skin surface with the unused disinfecting agent, discarding the unused disinfecting agent after use, and preparing another unused disinfecting agent for use.

The foregoing and/or other aspects of the present invention can likewise be achieved by providing an accessory configured to be attached to a medical device to disinfect a surface of the medical device and a skin surface, the accessory comprising a housing carrying a disinfecting material and including a thread that is configured to be attached to the medical device, wherein the disinfecting material is configured to disinfect the surface of the medical device and the skin surface.

Finally, the foregoing and/or other aspects of the present invention can be achieved by providing a method for disinfecting a skin surface and a surface of a medical device, the method comprising attaching an accessory to the device, disinfecting a surface of the medical device during the attachment, and disinfecting the skin surface with the accessory.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 is a perspective view of an assembled medication delivery pen of the prior art;

FIG. 2 is an exploded perspective view of the components of the medication delivery pen of FIG. 1 and of a pen needle;

FIG. 3 is a front elevation, partial section view of a first exemplary embodiment of an accessory attached to a cap of a medication delivery pen;

FIG. 4 is a front elevation view of a second exemplary embodiment of an accessory attached to a cap of a medication delivery pen;

FIG. 5 is a front elevation view of a third exemplary embodiment of an accessory attached to a cap of a medication delivery pen;

FIG. 6 is a front elevation view of a medication delivery pen and partial sectional view of a fourth exemplary embodiment of an accessory attached to a cap of the medication delivery pen;

FIG. 7 is a schematic drawing of the electrical components within the accessory of FIG. 6 without user input;

FIG. 8 is a schematic drawing of the electrical components within the accessory of FIG. 6 with user input;

FIG. 9 is a front elevation, partial sectional view of a fifth exemplary embodiment of an accessory removably attached to the medication delivery pen;

FIG. 10 is a rear perspective view of the accessory of FIG. 10 used to disinfect a skin surface; and

FIG. 11 is a front perspective view of the accessory of FIG. 1I with a seal on both ends.

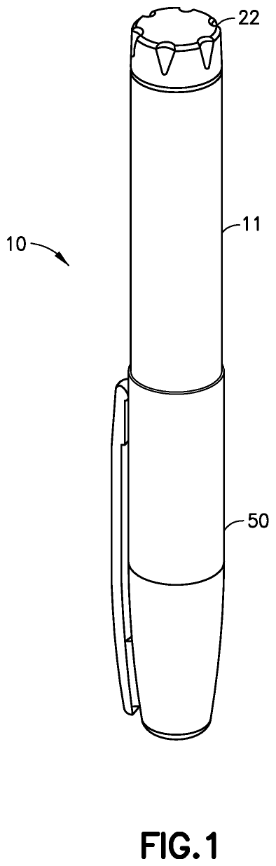

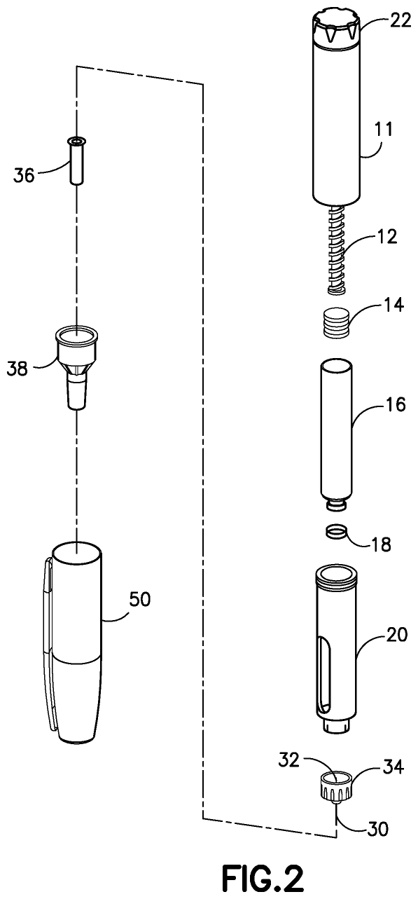

### DETAILED DESCRIPTION OF EXEMPLARY EMBODIMENTS

FIG. 3 illustrates a cap 50 for a safety device such as a catheter or a medical device such as a medication delivery pen 10 in accordance with an embodiment of the present invention. The embodiments of the cap 50 disclosed herein are most commonly configured to mount on to the medication delivery pen 10 without the pen needle present. However, with appropriate modifications, other types of medical devices such as needleless IV connectors, extension sets, IV sets, catheters, syringes (such as pre-fillable syringes), medication (e.g. insulin) vials, and other devices with externally accessible surfaces such as a septum that require disinfection can incorporate the cap 50 or otherwise be attachably connected to any exemplary accessory described below for disinfection purposes. Any surface or portion of the medical device (a portable medical device surface, for example) and/or a skin surface can be disinfected.

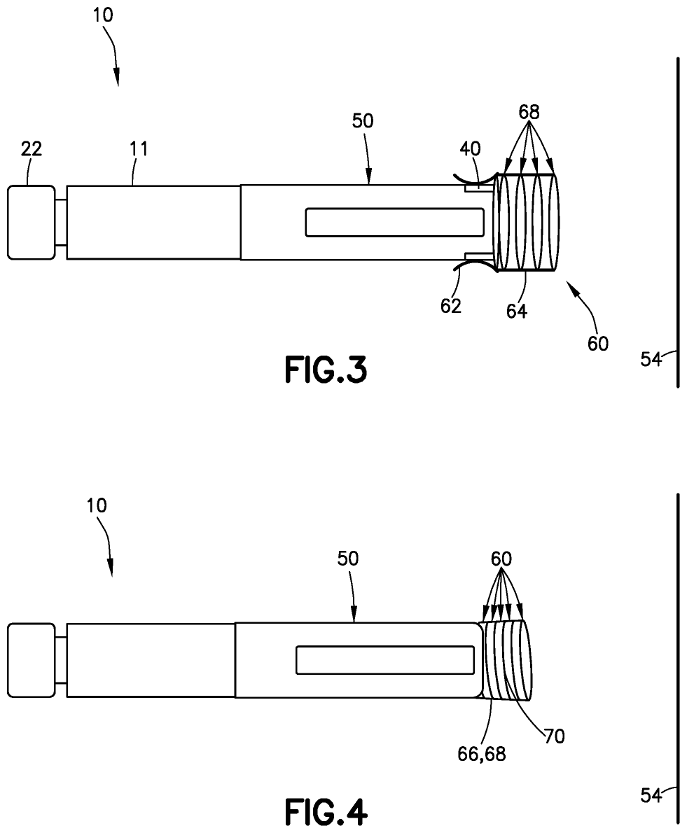

The medical device 10 such as the medication delivery pen is configured to be connectable to an exemplary accessory 60 either indirectly via a universal fitting 40 (illustrated in FIG. 3) or directly without the universal fitting 40 (illustrated in FIGS. 4-6 and 10). However, the universal fitting 40 can be sized for use in all disclosed embodiments herein.

Exemplary embodiments of the universal fitting 40 include a ring that tightens the fit between a proximal end of the accessory 60 and the medicament cartridge 16 of the medication delivery pen 10. A rotating sleeve that reduces the inner diameter when rotated and acts similarly to a telescoping pole is another universal fitting 40 that tightens the fit between the accessory 60 and the medication delivery pen 10. Further, using ribs, pleats, or scales as the universal fitting 40 provides an expandable, contractible and/or friction surface at the interface between the proximal end of the accessory 60 and the body 20. The universal fitting 40 can have prongs to provide a mechanical engagement between the accessory 60 and the body 20. Finally, another embodiment of the universal fitting 40 is a spring-loaded member that provides an applied force between the proximal end of the accessory 60 and the medication delivery pen 10.

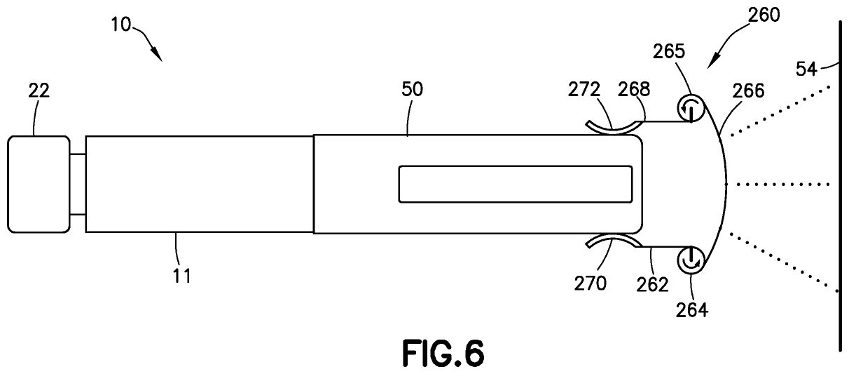

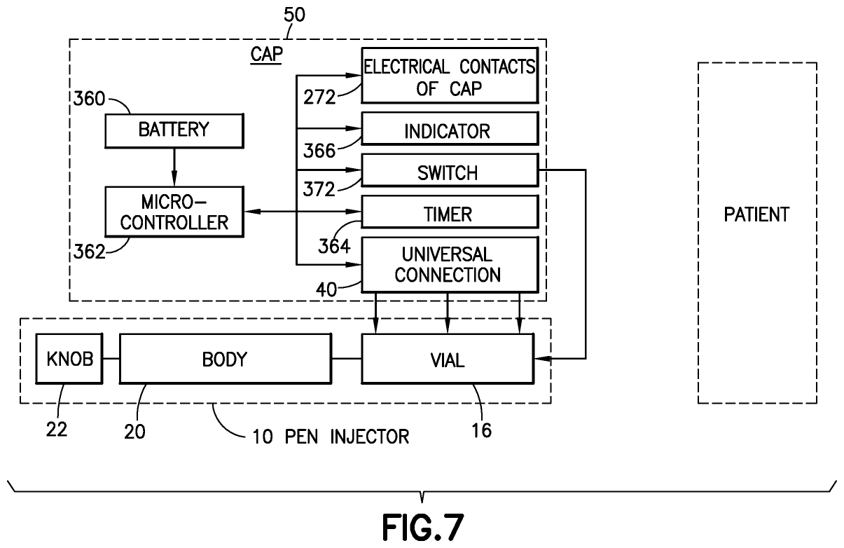

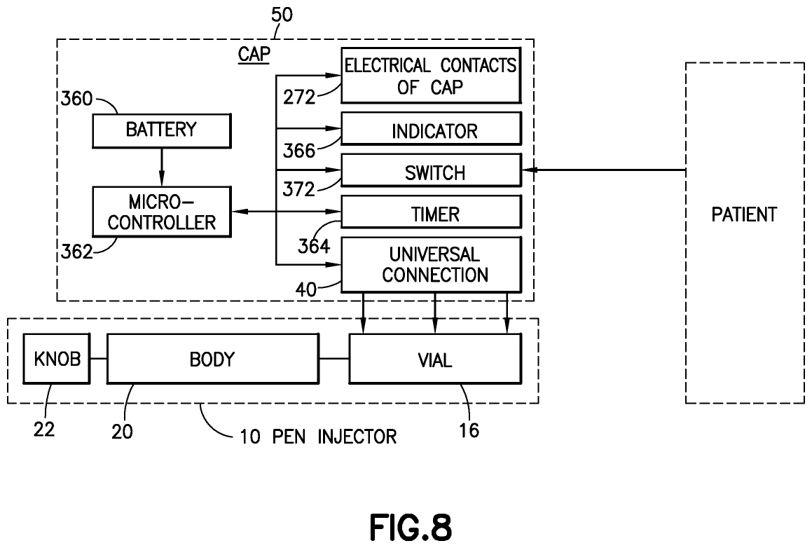

Status of the use of the universal fitting 40 is provided as feedback to a microcontroller 362, as described further below and as illustrated in FIGS. 7 and 8. Status of the use of the universal fitting 40 includes, for example, an engaged and unengaged position between the medication delivery pen 10 and the accessory 60. Alternately, the universal fitting 40 can be used without the cooperation of the microcontroller 362 as described further in FIG. 6.

The universal fitting 40 can also cooperate with the microcontroller 362 to vary commands for operating an accessory 60 based on the status. For example, when the universal fitting 40 and the accessory 60 are engaged, the microcontroller 362 issues a command to provide electrical energy to the accessory 60. On the other hand, if one or both of the connections are disengaged, the microcontroller 362 does not issue a command to provide electrical energy.

FIGS. 3 and 4 illustrate the accessory 60 being removably attached to the cap 50 of the medication delivery pen 10 to disinfect the skin surface prior to medication delivery. Specifically, all the accessory 60 embodiments disclosed herein can be used with any device including any of the medical devices 10 identified above, as well as a syringe with or without a syringe shield and a medication delivery pen with or without a cap 50. The accessory 60 in the embodiments of FIGS. 3 and 4 includes a mounting mechanism 62, a housing 64, a plurality of container 66, a disinfecting unit 68 and an interlocking mechanism 70.

The mounting mechanism 62 illustrated in FIG. 3 and other various embodiments advantageously attaches the accessory 60 to the medication delivery pen 10 for one time use or multiple use. FIG. 3 illustrates the mounting mechanism 62 as comprising a mechanical clip. However, other exemplary mounting mechanisms 62 can include a universal cap similar to the universal fitting described above, a spring loaded locking mechanism, a press fit, adhesion, hook and loop fasteners (Velcro), a threaded member, a spring clip and a button, for example. The mounting mechanism 62 is advantageously selected and/or sized based on the specific device 10 being used.

The disinfecting unit 68 described in embodiments disclosed herein includes one of a material, a surface, a substance, or a combination of substances as understood by one skilled in the art. The disinfecting unit 68 is configured to disinfect the skin surface or the medical device surface. The disinfecting unit 68 is further described below.

FIG. 3 illustrates the accessory 60 including the housing 64 that carries a plurality of disinfecting materials 68 stacked up in the housing 64. The housing 64 is preferably cup shaped and composed of a rigid material, plastic or metal, for example. In one embodiment, the housing 64 includes a spring (not shown) to move the plurality of disinfecting materials 68 to a distal end of the accessory 60 so that the user can disinfect the skin surface 54 while holding the medication delivery pen 10. In another embodiment, a user removes each selected disinfecting material 68 from the housing 64 for disinfecting the skin surface 54.

The housing 64 includes one or more notches (not shown) at its distal end that secures the plurality of disinfecting materials 68 in the housing 64. Specifically, the user would fold, bend or otherwise deform the selected disinfecting material 68 to avoid the notch for removal from the housing 64. Alternately, the one or more notches is flexible and flexes outwardly when removing one of the disinfecting materials 68. Although a notch is disclosed, although other commonly known means of retention are contemplated.

The disinfecting materials 68 used in the embodiments disclosed herein are widely available and commonly used in healthcare to promote injection site disinfection, such as disinfection scrubs, surgical scrubs or sponges. These materials provide a scrubbing action which improves disinfection practices. The disinfecting materials 68 can also be soaked in an alcohol solution or an iodine solution, for example, to provide more effective disinfection.

When the accessory 60 is mounted to the medication delivery pen 10, a disinfecting material 68 is applied to the skin surface 54 for disinfection. After use, the disinfecting material 68 is removed and a subsequent disinfecting material 68 is available for use.

FIG. 4 illustrates another embodiment where the accessory 60 includes the plurality of containers 66 instead of the housing 64 as disclosed in the embodiment of FIG. 3. The plurality of containers 66 each carry one of the plurality of disinfecting materials 68. Each of the plurality of containers 66 comprises a hollow disk for carrying one of the disinfecting materials 68 and a central opening (not shown) that exposes a central portion of the disinfecting material 68 so that a user can apply the disinfecting material 68 to the skin surface 54 for disinfection.

Alternately, in another embodiment, each of the plurality of containers 66 is a two piece assembly that can be opened to remove the disinfecting material 68 for disinfection. Specifically, the user can remove the selected container 66 and disassemble to remove and use the disinfecting material 68. The user could also disassemble the selected container 66 while it's attached to an adjacent container 66 and hold the medication delivery pen 10 to apply the disinfecting material 68 to a skin surface 54.

The plurality of containers 66 include an interlocking mechanism 70 that advantageously allows each of the containers 66 to removably connect to an adjacent container 66 or to the medication delivery pen 10. In this manner, the plurality of containers 66 is advantageously stacked for compactness and convenient use. Specifically, the plurality of containers 66 do not take up much space and are easy to carry instead of carrying multiple individual wipes. Also, the plurality of containers 66 are advantageously stored together and secured for easy removal and use.

The preferred interlocking mechanism 70 includes a snap fit such a protrusion and a cavity. Specifically, a front and rear surface of the each of the plurality of containers 66 include one of the protrusion and the cavity and the opposing surface includes the other of the protrusion and the cavity. The medication delivery pen 10 is configured to include a protrusion or a cavity to mate with the interlocking mechanism 70.

Alternately, the medication delivery pen 10 incorporates the universal connector 40 to similarly provide a detachable connection between the medication delivery pen 10 and one of the plurality of containers 66. Other interlocking mechanisms 70 include a flange engaging an angled opening that creates increasing friction as the two adjacent containers 66 move toward each other, press fit frictional forces, and various other male/female connections. Other undisclosed interlocking mechanisms 70 are contemplated by one skilled in the art.

When the accessory 60 is mounted to the medication delivery pen 10, the disinfecting material 68 in a selected container 66 is applied to the skin surface 54 for disinfection. After use, the selected container 66 is removed and a subsequent container 66 is available for use.

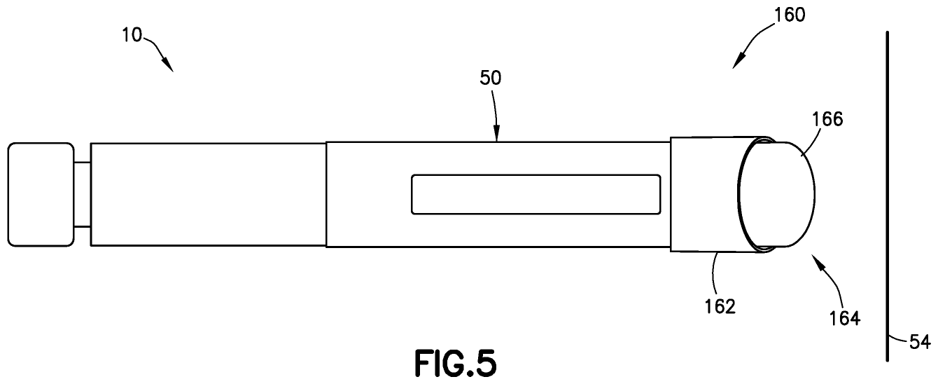

FIG. 5 illustrates another exemplary embodiment of an accessory 160 attached to the medication delivery pen 10. The accessory 160 includes a housing 162 and an applicator 164 comprising a disinfecting material 166. The housing 162 includes a mounting mechanism that is detachably connected to the medication delivery pen 10 via friction. Specifically, an inner diameter of the housing 162 engages an outer diameter of the medication delivery pen 10 via friction, for example. The housing 162 carries the applicator 164 and may optionally carry a disinfecting agent for use through the applicator 164 via the disinfecting material 166. The disinfecting agent can include, for example, a solid gel, an alcohol solution or an iodine solution as similarly described above. The disinfecting agent cooperates with the disinfecting material 166 of the applicator 164 to disinfect the skin surface.

The disinfecting material 166 of the applicator 164 can be a soft applicator, for example, a pad soaked in the disinfecting agent. Alternately, the disinfecting material 166 of the applicator 164 can be a hard applicator, for example, a ball point pen type of applicator that slowly releases the disinfecting agent carried by the housing 162. Another type of applicator 164 is a dauber tip.

For each of these embodiments of the applicator 164, the disinfecting agent can be soaked into the disinfecting material 166 of the applicator 164 and/or a gel or solution is stored in the housing 162 to periodically apply the disinfecting agent to the disinfecting material 166 of the applicator 164. The disinfecting material 166 is similar to the disinfecting material 68 described above. The disinfecting material 166 cooperates with the disinfecting agent in the housing 162 to allow the user to disinfect the skin surface 54.

In an alternate embodiment, the applicator 164 attaches directly to the medication delivery pen 10 via a mounting mechanism (similarly illustrated in FIG. 3) without the housing 162. In this embodiment, the disinfecting material 166 of the applicator 164 is soaked with the disinfecting agent.

FIG. 6 illustrates an exemplary embodiment of an accessory 260 including a housing 262, one or more rolls 264, a disinfecting material 266 and a mounting mechanism 268 provided with electrical contacts 270 that are configured to engage electrical contacts 272 of the medication delivery pen 10. The housing 262 is connected to the mounting mechanism 268 for mounting on the medication delivery pen 10. Specifically, the housing 262 is removably attached to a cap 50 of the medication injection pen 10 via the mounting mechanism 268 as similarly described above.

The housing 262 is configured to carry one or two rolls 264, 265, also referred to as cylinders, disposed on alternate sides of the accessory 260. The cylindrical shape of the one or two rolls 264, 265 includes pins, an oval or an oblong shape, for example, being solid or hollow in volume. One skilled in the art would understand that a variety of symmetrical shapes could be used as the one or two rolls 264, 265.

One roll 264 is wrapped by a strip of unused disinfecting material 266. The strip of unused disinfecting material 266 includes a disinfecting agent applied to a cleaning material as similarly described above. The unused disinfecting material 266 is advanced across the distal end face of the accessory 260 to disinfect the skin surface 54. Specifically, the unused disinfecting material 266 is unwrapped from the roll 264 at a distal end of the accessory 260.

In another embodiment, the strip of unused disinfecting material 266 includes a plurality of disinfecting materials 266 connected together for individual use. In other words, the strip of unused disinfecting materials 266 can comprise a carrier strip that includes multiple disinfecting sites to apply to the skin surface 54. This configuration would operate similarly as above.

In one embodiment, a proximal side of the disinfecting material 266 includes an adhesive to adhere to the housing 262 so that the user can hold the medication delivery pen 10 and disinfect the skin surface 54 using the disinfecting materials 266. In another embodiment, the housing 262 provides a locking mechanism (not shown) that holds the disinfecting materials 266 in place so that the user can hold the medication delivery pen 10 and disinfect the skin surface 54.

After the disinfecting material 266 is used for disinfecting the skin surface 54, the used disinfecting material 266 is discarded by the user. The user can then unwrap and advance a new length of unused disinfecting material 266 from the roll 264. The new length of disinfecting material 266 is now positioned and ready for disinfecting the skin surface 54.

In another embodiment, after the selected disinfecting material 266 is used, the selected disinfecting material 266 is wrapped around the second roll 265. At the same time, the new length of disinfecting material 266 is unwrapped from the first roll 264 and is now ready for disinfecting the skin surface 54. This process can be performed manually or automated similar to an automated toilet seat cover or an audio cassette, for example.

The specific positions of the first and second rolls 264, 265 described herein are exemplary, as they can be disposed in a variety of positions to provide proper functionality as understood by one skilled in the art. For example, in yet another embodiment, the first and second rolls are concentric. Specifically, the first and second rolls 264, 265 are adjacent and concentric to each other on one side of the accessory 260. A rod is disposed on the side opposing the side where the first and second rolls 264, 265 are disposed. The rod is used to route the disinfecting material 266 from the first roll 264 to the second roll 265.

When the accessory 260 is assembled, the unused disinfecting material 266 exits the first roll 264, wraps around the rod and engages the second roll 265. The rod is used to spread the unused disinfecting material 266 and provide a cleaning surface. The disinfecting material 266 disinfects the skin surface 54 upon use. Afterwards, the used disinfecting material 266 travels around the rod to the second roll 265. The travel path from the rod to the second roll 265 is disposed proximal to the travel path of the unused disinfecting material 266 between the first roll 264 and the rod. This orientation prevents the used disinfecting material 266 from contacting the surface to be disinfected. While the used disinfecting material 266 travels to the second roll 265, another unused disinfecting material 266 exits the first roll 264 and the accessory 260 is once again ready for disinfecting. This process repeats for each use while the second roll 265 accumulates all the used disinfecting material 266.

The automated accessory 260 further includes wiring connecting the electrical contacts 270 to a motor (not shown) for rotating the second roll 265. Rotation of the second roll 265 controls movement of the unused disinfecting material 266 from the first roll 264 into position for disinfection and controls movement of the used disinfecting material 266 after disinfection for wrapping around the second roll 265.

FIGS. 7 and 8 further illustrate the medication delivery pen 10 that provides electrical energy to automate the accessory 260. Specifically, the cap 50 includes a power source 360 that provides power to the cap 50. The power source 360 is preferably a flexible battery that wraps along an inner surface of a sidewall. The power source 360 can also be a lithium battery. Finally, the power source 360 can be wired circuitry that provides power (AC/DC current) to the cap 50.

If the power source 360 is a battery, the battery 360 can be rechargeable via solar energy, motion or electricity (wired or wireless). Alternatively, or in addition, the battery 360 can be discarded and replaced. Further, the cap 50 can be replaced when the battery 360 is depleted. The power source 360 can be disposed on the inner or outer surface of the sidewall or top wall.

As illustrated in FIGS. 7 and 8, the power source 360 is configured to specifically provide power to a microcontroller 362 of the cap 50 (see FIG. 7) or directly to the electrical contacts 272 of the cap. The electrical contacts 272 of the cap electrically communicate with the electrical contacts 270 of the accessory 260 to automate the operation of the first and second rolls 264, 265 as described above.

The microcontroller 362, as commonly understood by one skilled in the art, is programmed to sense and control the operation of the accessory 260. Specifically, the microcontroller 362 receives feedback and issues commands to various components of the medication delivery pen 10 and the accessory 260 including, for example, the universal fitting 40 (as described above), the electrical contacts 272 of the cap 50 (as described above), a timer 364, an indicator 366 and a switch 372.

The command that controls the operation of the accessory 260 is received from the microcontroller 362 or directly from the switch 372 (see FIG. 8). The cap 50 further includes the switch 372 that causes the microcontroller 362 to generate commands that activate and deactivate the accessory 260. Alternately, or in addition, as illustrated in FIG. 8, the switch 372 itself connects and disconnects the power source 360 to the accessory 260. The switch 372 is disposed on the inner surface of the sidewall of the cap 50. However, the switch 372 can be disposed on any inner or outer surface of the cap 50. The switch 372 can be an actuated switch such as a micro switch, a spring-loaded switch, or a button switch. In another embodiment, the switch 372 includes first and second switches to separately activate and deactivate the first roll 264 and the second roll 265, respectively, of the accessory 260.

Specifically, the micro switch and/or the spring-loaded switch can be activated based on pressure from the user prior to injection (manual) or an exerted force between the cap 50 and the accessory 260 during assembly (automatic). As illustrated in FIG. 8, upon sensing an increased pressure, the micro switch 372 sends a signal to the microcontroller 362 to activate the accessory 260. When the user releases the pressure or when the medication delivery pen 10 and the accessory 260 are disassembled, the pressure is lessened and the micro switch 372 sends a signal to the microcontroller 362 to deactivate the accessory 260.

The spring force therefore provides a one-time activation of the accessory 260. After a predetermined time period, the accessory 260 is deactivated. Alternatively, a manual switch could be implemented to trigger the activation of the accessory 260 for a desired duration prior to each use. The activation and deactivation of the accessory 260 in this regard can be automatic, instantaneous or alternate based on the programmed signaling from the microcontroller 362 or the engagement and disengagement of the micro switch 372.

The switch 372, if provided as a spring-loaded switch, can release a spring force upon receiving an increased pressure during assembly of the medication delivery pen 10 to the accessory 260. The spring force provides a one-time activation of the accessory 260. After a predetermined period of time, the accessory 260 is deactivated.

The timer 364 can be incorporated into the spring-loaded switch 372, for example, to provide a predetermined time period of use of the accessory 260 or a time delay before use of the accessory 260. The timer 364 can be activated when the switch 372 is engaged. For example, the timer 364 can cause the accessory 260 to wait for up to 120 seconds before a new length of unused disinfecting material 266 is unwrapped. The timer 364 can also cooperate with the microcontroller 362 to vary commands for activating and deactivating the accessory 260.

As illustrated in FIG. 8, the switch 372, when provided as a button switch, can deflect, release a force and/or establish electrical contact with the microcontroller 362 and/or the accessory 260 based on an operation, such as a depression, for example, by a user such as the clinician or patient. In this manner, the user is able to control the activation and deactivation of the accessory 260.

The switch 372 can also be a proximity sensor, a Hall Effect sensor, a photo sensor, an optical sensor and a force sensor. The operation of these sensors is commonly understood by one skilled in the art. The proximity sensor can sense that the accessory 260 is connected to the medication delivery pen 10 and instructs the microcontroller 362 of this condition. Subsequently, the microcontroller 362 can command the activation and operation of the accessory 260. When the accessory 260 is removed from the medication delivery pen 10, the proximity sensor informs the microcontroller 362 of this condition and the microcontroller 362 commands the accessory 260 to stop operation.

The cap 50 further includes an indicator 366 that displays a plurality of conditions such as indicating when the accessory 260 is activated or deactivated, how many disinfecting materials 266 have been used, how many disinfecting materials 266 are unused, when the disinfecting/sterilizing process of the medication delivery pen 10 or skin surface 54 is complete, and the remaining life of the power source 360. The indicator 366 communicates with the microcontroller 362 to receive a status of one or more of these conditions prior to display. The indicator 366 displays these conditions via a plurality of media commonly known by those skilled in the art such as, for example, colors, symbols and text.

In a simpler implementation as mentioned above, the button switch 372 can control the electric power from the power source 360 directly to the accessory 260, without the microcontroller 362 or timer 364. In this case, the user controls the power to the accessory 260 by depressing and releasing the button switch 372. That is, when the switch 372 is in operation, or depressed, the accessory 260 uses the electric power from the power source 360 for operation. When the switch 372 is not in operation, or not depressed, the accessory 260 does not use the electric power from the power source 360.

The mounting mechanism 268 or mechanical clip elastically compresses the cap 50 of the medication delivery pen 10 to secure the accessory 260 onto the medication delivery pen 10. In one embodiment, a proximal end portion of the mechanical clip 268 includes electrical contacts 270 that engage electrical contacts 272 of the medication delivery pen 10 at the compressed section. In this manner, electrical power is transmitted from the power source 360, through the electrical contacts 270, 272 and through electrical wiring disposed in the hollow mechanical clip 268 to electrically connect to and provide energy to the accessory 260.

In another embodiment, the power source 360 is an independent, stand-alone battery such as a battery commonly used for a watch or a rechargeable battery. Accordingly, the power source 360 provides electrical energy to the timer 364 and switch 372.

The accessory 260 advantageously provides a single accessory with multiple disinfectant materials that is easier and more convenient to carry than multiple antiseptic wipes. The accessory 260 is advantageously carried with the medication delivery pen 10 for convenient use. Also, the accessory 260 promotes good injection practices because the disinfecting material 266 is readily available for use at the time of injection and the used disinfecting material 266 is discarded and prevents the user from using it multiple times.

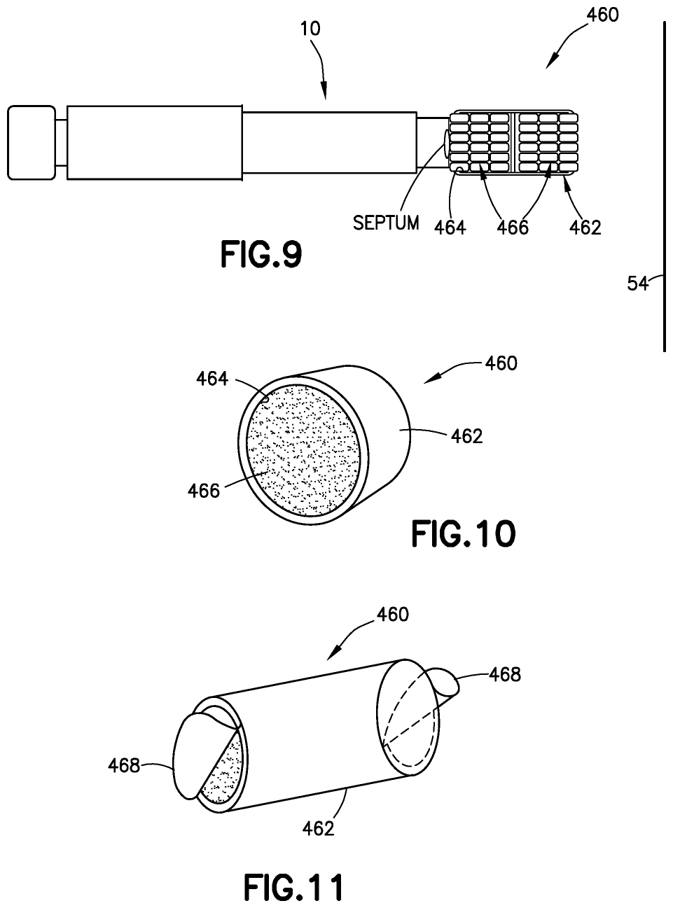

FIGS. 9-11 illustrate another exemplary embodiment of an accessory 460 that is configured to disinfect a surface of a medication delivery pen 10 and a skin surface. Specifically, the accessory 460 includes a housing 462 having inner threads 464, a disinfecting material 466 and a seal 468. The inner threads 464 are disposed at a proximal end of the housing 462 and are configured to engage, for example, threads of a medication delivery pen 10 or threads of a vial.

The housing 462 comprises a cylindrical shape and carries the disinfecting material 466. The disinfecting material 466 is disposed to slightly protrude from a distal end of the housing 462. The inner threads 464 overhang from a proximal end of the disinfecting material 466 so that the inner threads 464 are advantageously engaged before disinfecting begins. Such a configuration also protects the disinfecting material 466 before use. The disinfecting material 466 is similarly described in the embodiments above.

During assembly, as the inner threads 464 threadably engage the threads of the medication delivery pen 10 or vial, the disinfecting material 466 advantageously disinfects a septum 18 of the medication delivery pen 10 or vial. In particular, engagement and disinfecting advantageously occur simultaneously. Further, the rotating movement during the threaded engagement between the accessory 460 and the medication delivery pen 10 or vial advantageously provides a scrubbing action to the septum 18 of the medication delivery pen 10 which further improves the disinfection. Furthermore, the scrubbing action received by the septum 18 is controlled by the threaded engagement so that the septum 18 is disinfected effectively and consistently. Finally, after the accessory 460 is secured to the medication delivery pen 10, the user can disinfect the skin surface 54.

The accessory 460 is provided for single use. Prior to use, the disinfecting material 466 in the housing 462 of the accessory 460 is sealed at the proximal and distal ends by a seal 468, such as a peel tab. Thus, the seal 468 preserves sterility of the disinfecting material 466 prior to use.

Disinfection of the septum 18 during assembly of the accessory 460 is advantageously convenient and one less thing for the user to think about prior to injection. An additional advantages that the accessory 460 provides is that the accessory 460 alone disinfects multiple surfaces (i.e. the septum 18 and the skin surface). Thus, the user is not required to carry multiple wipes or disinfecting materials for disinfection of multiple surfaces.

In another embodiment, the accessory 460 includes a disinfecting liquid agent (not shown) sealed in a cavity (not shown) of the housing 462 and a movable piercing member (not shown) also disposed in the housing 462. When the housing 462 is threadably engaged to the medication delivery pen 10, the piercing member moves to pierce the sealed cavity, allows the disinfecting liquid agent to be released from the cavity and allows the disinfecting liquid agent to be absorbed by the disinfecting material 466. Accordingly, after a period of time, the disinfecting material 466 is soaked in the disinfecting liquid agent to further improve the effectiveness in disinfecting the skin surface and the septum 18 of the medication delivery pen 10.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. The specification describes specific examples to accomplish a more general goal that may be accomplished in another way.

As used in this application, the terms “front,” “rear,” “upper,” “lower,” “upwardly,” “downwardly.” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

What is claimed is:

1. An accessory configured to be attached to a medical device to disinfect a skin surface or a surface of the medical device, the accessory comprising:
a plurality of disinfecting materials; and
a mounting mechanism that is configured to attach and detach the accessory to the medical device.

2. The accessory of claim 1, wherein the medical device comprises one of a medication delivery pen, a syringe, a patch pump and a safety device.

3. The accessory of claim 1, wherein
the medical device comprises one of a medication delivery pen having a pen cap and a syringe having a syringe shield; and
the mounting mechanism is attached to one of the pen cap and syringe shield.

4. The accessory of claim 1, wherein the mounting mechanism comprises one of a universal cap, spring loaded member, press fit, adhesive, threads and a button.

5. The accessory of claim 1, further comprising:
a housing that carries the plurality of disinfecting material; wherein
the mounting mechanism is connected to the housing.

6. The accessory of claim 1, further comprising:
a plurality of containers each carrying one of the plurality of disinfecting materials; and
an interlocking mechanism configured to removably connect adjacent containers to each other and configured to removably connect one of the containers to the medical device.

7. An accessory configured to be attached to a medical device to disinfect a skin surface or a surface of the medical device, the accessory comprising:
an applicator including disinfecting material; and
a mounting mechanism that is configured to attach and detach the applicator to the medical device.

8. The accessory of claim 7, further comprising
a housing that engages the applicator to enclose the disinfecting material; wherein
the housing comprises the mounting mechanism.

9. The accessory of claim 7, wherein the applicator includes a dauber tip that is soaked with the disinfecting material.

10. An accessory configured to be attached to a medical device to disinfect a skin surface or a surface of the medical device, the accessory comprising:
a cylinder wrapped by a strip of disinfecting material; and
a mounting mechanism that is configured to attach and detach the cylinder to the medical device; wherein
the strip of disinfecting material is unwrapped to disinfect the skin surface or the surface of the medical device.

11. The accessory of claim 10, further comprising:
a housing carrying the cylinder; wherein
the cylinder comprises
a first cylinder configured to be wrapped by a strip of unused disinfecting material; and
a second cylinder configured to be wrapped by a strip of used disinfecting material.

12. The accessory of claim 11, wherein after a length of disinfecting material unwraps from the first cylinder and is used for disinfecting, the used disinfecting material wraps around the second cylinder.

13. The accessory of claim 12, wherein the used disinfecting material automatically wraps around the second cylinder.

14. The accessory of claim 12, wherein the mounting mechanism comprises electrical contacts that is configured to electrically connect to electrical contacts of the medical device to provide electrical power for operation of the accessory.

15. The accessory of claim 10, wherein
a length of unused disinfecting material is unwrapped from the cylinder for disinfecting; and
the used disinfecting material is subsequently manually discarded.

16. A method for disinfecting a skin surface or a surface of a medical device, the method comprising:
attaching an accessory to the medical device;
activating the accessory to provide an unused disinfecting agent;
disinfecting the skin surface or the surface of the medical device with the unused disinfecting agent;
discarding the used disinfecting agent after use; and
preparing another unused disinfecting agent for use.

17. The method of claim 16, wherein the discarding occurs automatically.

18. The method of claim 16, wherein the preparing occurs automatically.

19. An accessory configured to be attached to a medical device to disinfect a surface of the medical device and a skin surface, the accessory comprising:
a housing carrying disinfecting material and including a thread that is configured to be attached to the medical device; wherein
the disinfecting material is configured to disinfect the surface of the medical device and the skin surface.

20. The accessory of claim 19, further comprising:
a first seal disposed at a proximal end of the housing to cover the disinfecting material in the housing; and
a second seal disposed at a distal end of the housing to cover the disinfecting material in the housing.

21. The accessory of claim 19, wherein when the housing threadedly engages the medical device, the disinfecting material disinfects a septum of the medical device.

22. A method for disinfecting a skin surface and a surface of a medical device, the method comprising:
attaching an accessory to the medical device;
disinfecting a surface of the medical device during said attachment; and
disinfecting the skin surface with the accessory.

23. The method of claim 22, further comprising:
threading the accessory to the medical device during attachment; wherein
the surface of the medical device comprises a septum.
